(19) United States
(12) Patent Application Publication
(10) Pub. No.: US 2014/0346065 A1
(43) Pub. Date: Nov. 27, 2014

(54) DEVICE FOR WARMING WIPES

(71) Applicant: Kym Powell, (US)

(72) Inventor: Michael Chijoff, Footscray (AU)

(21) Appl. No.: 14/374,746

(22) PCT Filed: Jan. 25, 2013
(86) PCT No.: PCT/AU2013/000058

§ 371 Date: Jul. 25, 2014

(30) Foreign Application Priority Data
Jan. 25, 2012 (AU) 2012900290

(51) Int. Cl. B65D 83/08; A61F 7/00

(52) U.S. Cl. CPC B65D 83/0805; A61F 7/00
USPC 206/233

(57) ABSTRACT

There is disclosed herein a device for warming wipes, comprising a body configured to rest atop a pile of wipes so as to impart heat thereto, and being configured with an opening therethrough, through which successive uppermost wipes in the pile can be drawn.

### FIELD OF THE INVENTION

The present invention relates to a device for keeping moist napkins or towelettes warm, and to a napkin/towelette holder comprising such a device.

### BACKGROUND

Pre-moistened, disposable towelettes or napkins, commonly referred to as “wipes”, are popular for personal hygiene, particularly for cleaning babies and small children but also for hand washing and make-up removal.

Because the wipes are moist, they may feel cold when applied to the skin, particularly during winter. If the wipes are used for cleaning babies or the elderly or infirm, the cold feeling can cause shock and discomfort.

Containers which hold and heat wipes have been proposed but are generally complicated in their construction and operation.

### SUMMARY OF THE INVENTION

According to a first aspect of the present invention, there is provided a device for warming wipes, comprising a body configured to rest atop a pile of wipes so as to impart heat thereto, and being configured with an opening therethrough, through which successive uppermost wipes in the pile can be drawn.

Preferably, the opening comprises an aperture.

Preferably, the body is configured in the form of a block or pad.

Preferably, the body comprises a covering and a substance, contained in the covering, which can be heated so as to be able to output heat for warming the wipes. The covering may comprise a jacket, casing or shell.

Preferably, the substance comprises a phase change material.

In one preferred embodiment of the invention, the phase change material comprises Thermopol or PEG-4000.

In another preferred embodiment of the invention, the phase change material comprises a liquid from which a solid can nucleate whereby heat is output therefrom. Preferably, the device then further comprises a nucleation element, e.g. a disc, which can be manipulated to effect nucleation of the solid.

Preferably, phase change material comprises a solution containing a dissolved compound which can precipitate out of the solution and in so doing liberate heat, having a concentration which such that compound is fully dissolved when the body is sufficiently heated and can remain stably dissolved when the body cools to room temperature whereby the solution is supersaturated with the compound, and the body includes means to effect nucleation of the compound. Preferably, the means to effect nucleation comprises a nucleation element. Preferably, the solution comprises an aqueous sodium acetate solution.

In a further preferred embodiment of the invention, the substance comprises loose-fill material. Preferably, the loose-fill material comprises grains.

In a preferred embodiment of the invention, the device further comprises an insulating jacket or skirt received over the body to restrict heat loss therefrom.

Preferably, the insulating jacket or skirt is configured such that a lower face of the body is exposed to contact directly the uppermost wipe in the pile.

Preferably, the body/device is heatable in a microwave oven.

According to a second aspect of the present invention, there is provided an apparatus for storing and warming wipes, comprising:
a receptacle for holding a pile of wipes; and
a device according to any one of the preceding claims receivable atop the pile in the receptacle,
wherein the receptacle is configured to permit the successive uppermost wipes to be drawn through the opening and removed from the receptacle when the device is received atop the pile in the receptacle.

According to a preferred embodiment of the invention, the apparatus is provided with said pile of wipes held in the receptacle, and said device is arranged atop the pile in the apparatus. Preferably, the wipes are interleaved whereby withdrawal of an uppermost wipe in the pile through the opening effects drawing of part of the subsequent uppermost wipe through the opening.

Preferably, the device is configured to occupy substantially all of an area of an interior of the receptacle to be occupied by the pile of wipes which is normal to an upright axis through the interior.

### BRIEF DESCRIPTION OF THE DRAWINGS

The invention will now be described, by way of non-limiting example only, with reference to the accompanying drawings, in which:

FIG. 1A is a front three-quarter view of a wipes heating device according to a preferred embodiment of the present invention;

FIG. 1B is a bottom view of the device shown in FIG. 1A;

FIG. 1C shows a phase change material-filled casing which forms part of the device shown in FIG. 1A;

FIG. 2 is a front three-quarter view of a typical container for holding wipes, in a partially open condition;

FIG. 3 is a front three-quarter view of the container of FIG. 2 and the heating device of FIG. 1A received atop a pile of wipes therein;

FIG. 4 is a front three-quarter view of the arrangement of FIG. 3 in which an uppermost wipe of the pile in the container has been partially drawn through an opening through the heating device;

FIG. 5 is a front three-quarter view of the arrangement of FIG. 3 closed condition;

FIG. 6 is a front three-quarter view of a heating device according to an alternative preferred embodiment of the invention, and showing manipulation thereof to cause it to generate heat; and

FIG. 7 is a front thee-quarter view of a heating device according to another alternative preferred embodiment of the present invention;

FIG. 8A is a front three-quarter view of a heating device according to yet another alternative preferred embodiment of the present invention;

FIG. 8B is a top view of the device of FIG. 8A;

FIG. 8C is a bottom view of the device of FIG. 8A;

FIG. 8D shows views taken through sections A-A and B-B in FIG. 8B;

FIG. 9A is a front three-quarter view of a heating device according to still another alternative preferred embodiment of the invention, which comprises the device of

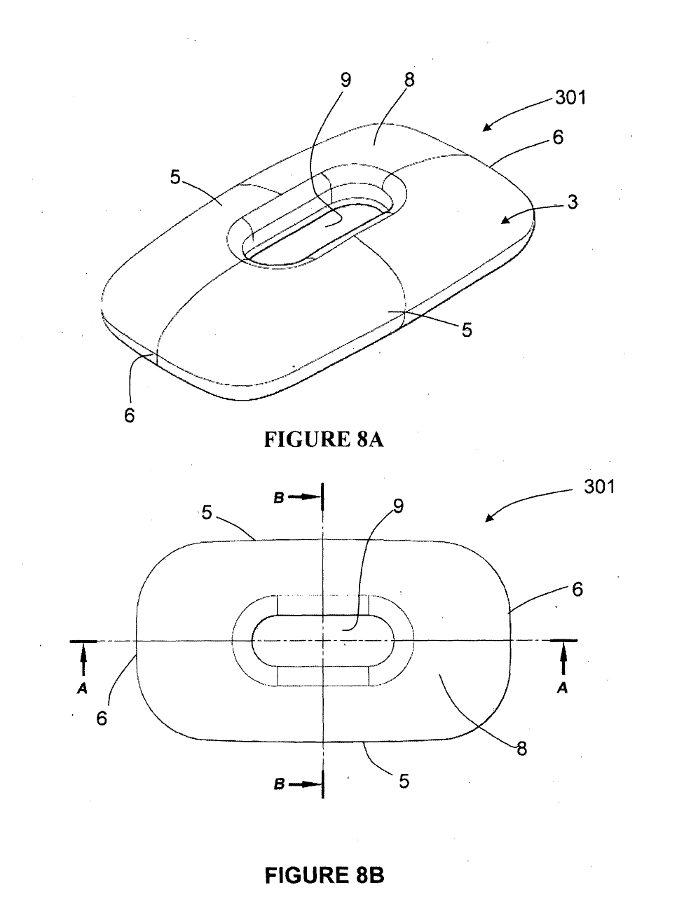

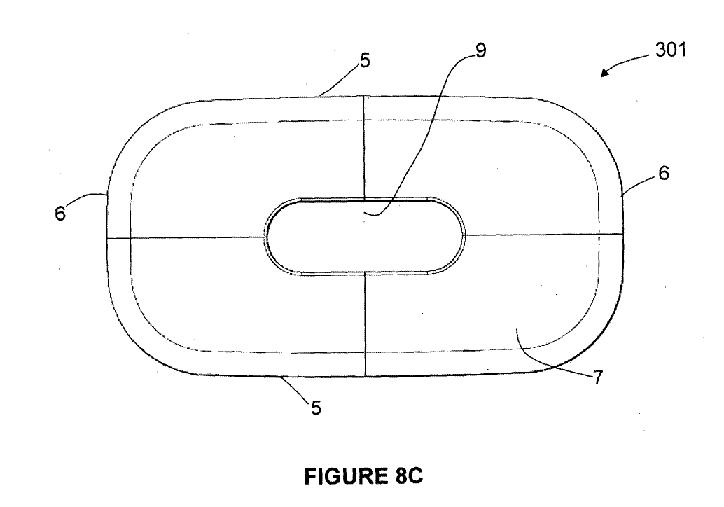

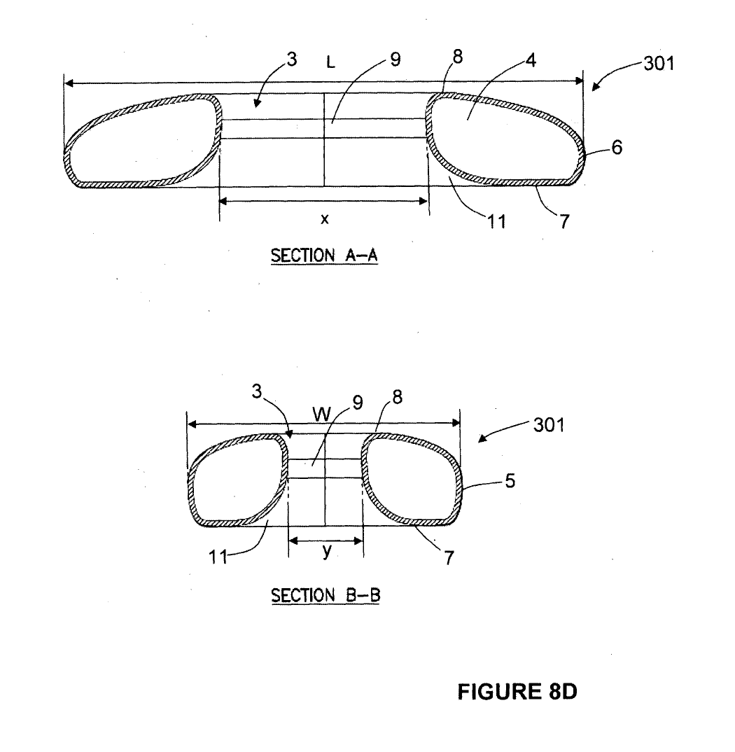

FIG. 8A and an insulating jacket received thereover;

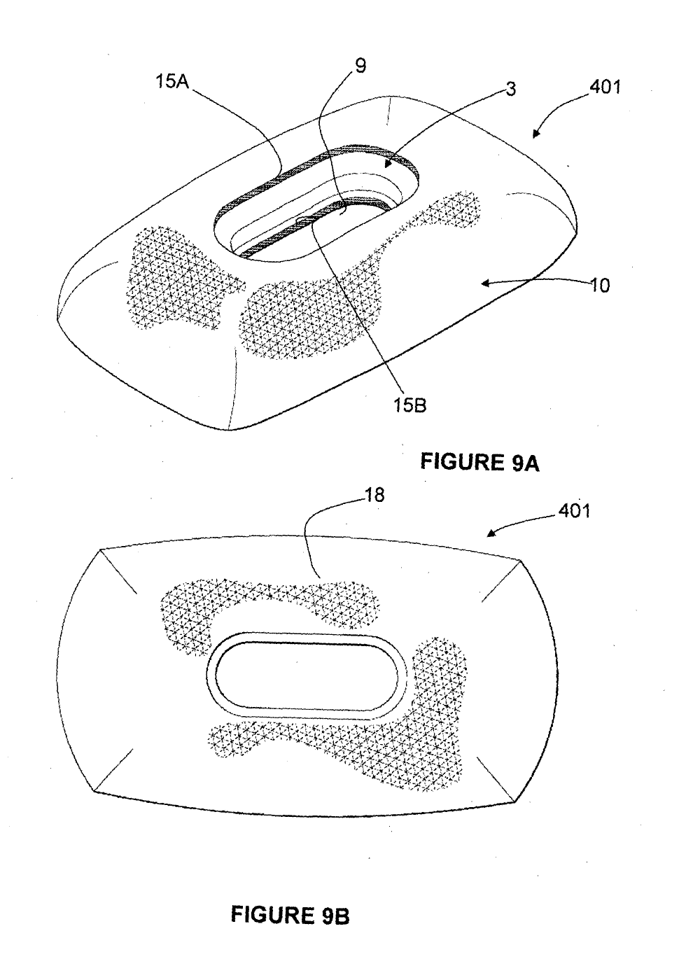

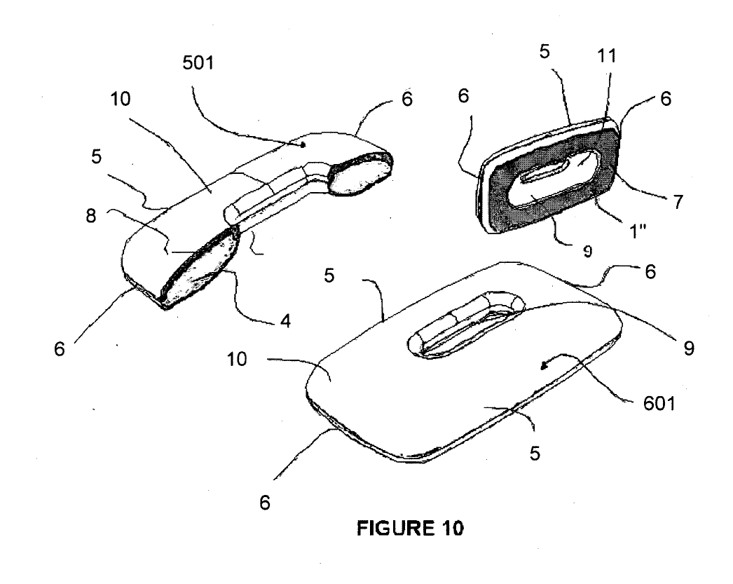

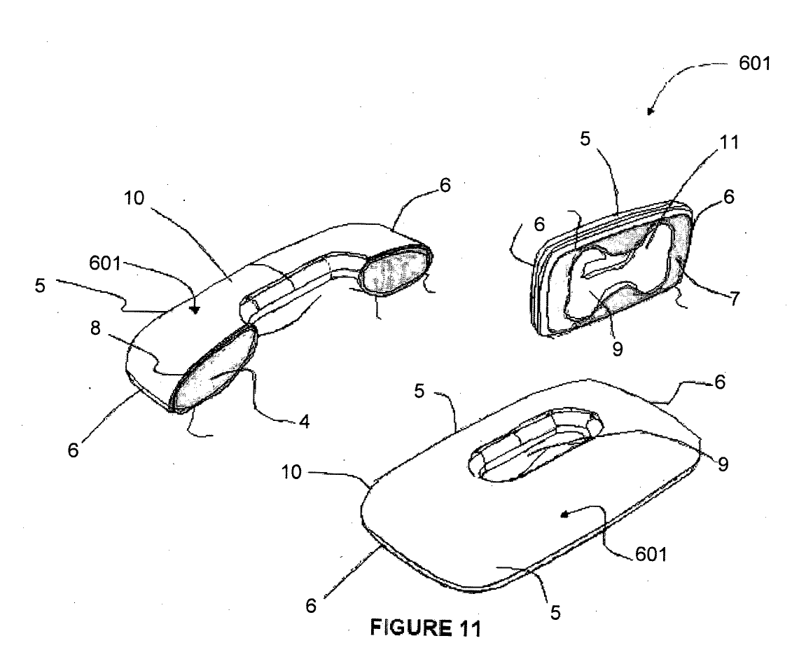

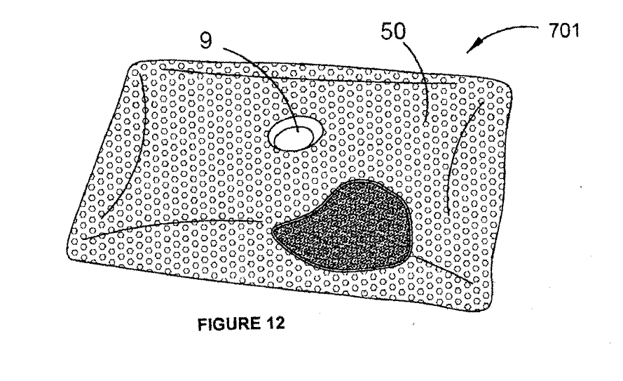

FIG. 9B is a bottom view of the device of FIG. 9A;

FIG. 10 shows details of a heating device according to a further alternative preferred embodiment of the present invention;

FIG. 11 shows details of a heating device according to another further alternative preferred embodiment of the present invention; and

FIG. 12 is a front thee-quarter view of a heating device according to yet a further alternative preferred embodiment of the present invention.

### DETAILED DESCRIPTION OF THE PREFERRED EMBODIMENTS

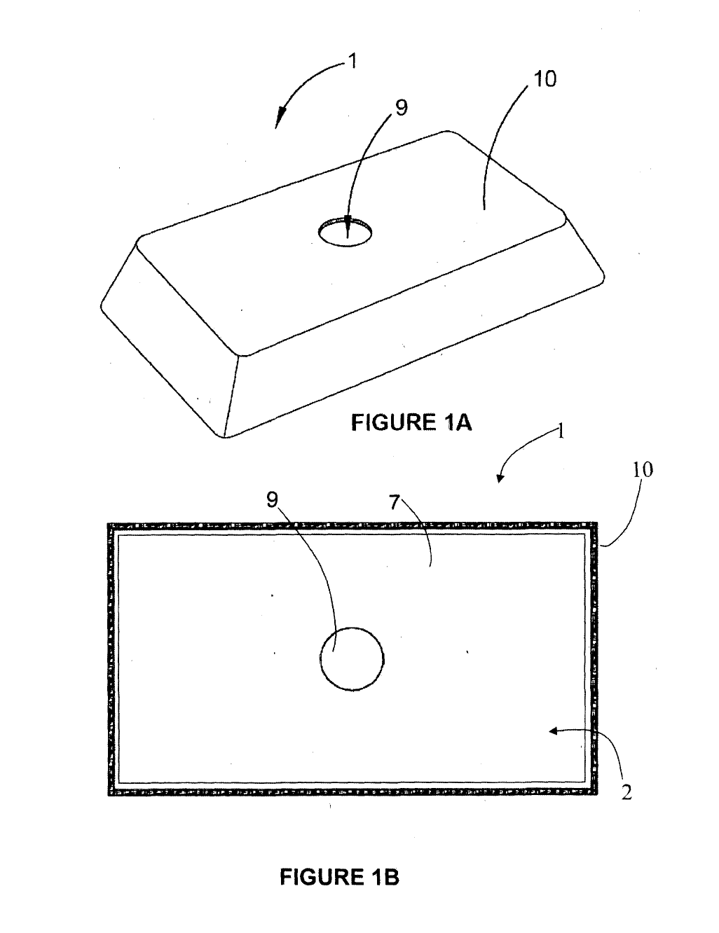

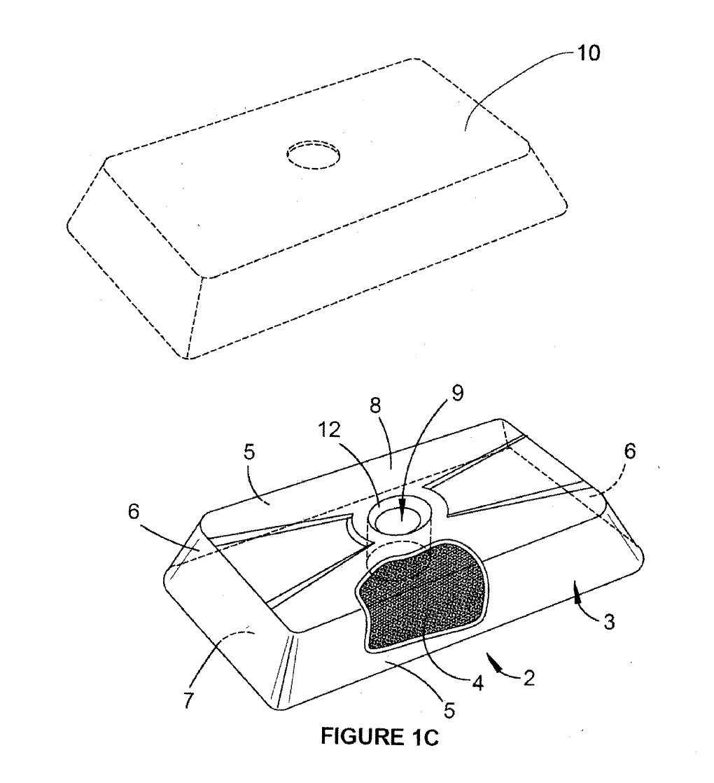

Shown in FIG. 1A is a microwave-safe device 1 in the form of a generally rectangular or oblong block or pad, for heating wipes according to a preferred embodiment of the invention. Referring to FIG. 1C, the device 1 comprises a heating block 2 which comprises a closed microwave-safe covering or casing 3 and a phase change material (PCM) 4 inside the casing 3, which material will described in further detail shortly, the covering 3 and PCM 4 defining a body. The casing 3 is rigid or semi-rigid and is blow-moulded from a suitable plastic such as polypropylene.

The casing 3 comprises side walls 5, end walls 6, a bottom wall or base 7, which is preferably flat such that substantially the entirety thereof will contact the wipes when it is received thereagainst, and a top wall 8. The casing 3 is configured with a centrally disposed opening, in the form of an aperture 9, which extends through the device 1, between the base 7 and top wall 8. The aperture 9 is bounded at its periphery by a circumferential wall 12 of the casing 3, which extends from the bottom wall 7 to the top wall 8. In the present embodiment, the aperture 9 has a circular cross-section, though openings/apertures having other cross-sectional configurations, including, for example, rectangular, oval and triangular configurations, are possible without departure from the invention (as will be clear from the disclosure, later herein, of further embodiments).

Referring also to FIGS. 1A and 1B, the device 1 further comprises a microwave-safe insulating jacket or skirt 10, which is received over the block 2, covering the top wall 8, side walls 5 and end walls 6, though not the base 7, and is formed with an opening therethrough which aligns with the aperture 9. The skirt 10 is formed of a resiliently flexible closed-cell foam material, preferably Neoprene/polychloroprene, though may alternatively be formed from any of a range of suitable insulating materials, and may, for example, be rigid, semi-rigid or pliable. The skirt 10 is removable from the block 2, and may be themed—e.g. it may carry an image which is pleasing to a baby or small child—and may be provided as part of a set of interchangeable skirts 10 which are themed differently/carry different images. Alternatively, the skirt 10 may be permanently attached to the block 2.

Generally speaking, a PCM, as will be known to a person skilled in the art, is a material used to store the latent heat which it absorbs when a phase change occurs therein. In the present embodiment, the PCM is one which melts or softens when heated and releases heat as it solidifies or hardens while cooling, with a net effect that cooling of the material is drawn out so that the PCM (and thus the device 1) remains warm for a sustained period (though alternative types of PCM may be employed without departure from the invention, as will be discussed later). The particular PCM 4 employed in the block 2 of this embodiment comprises polyethylene glycol (PEG), preferably “PEG-4000”, which is available as a proprietary product known as Thermopol. It has been found that this material, after being heated for a few minutes in a microwave oven, can remain at a temperature of 30° C. to 40° C. for as long as 7 hours.

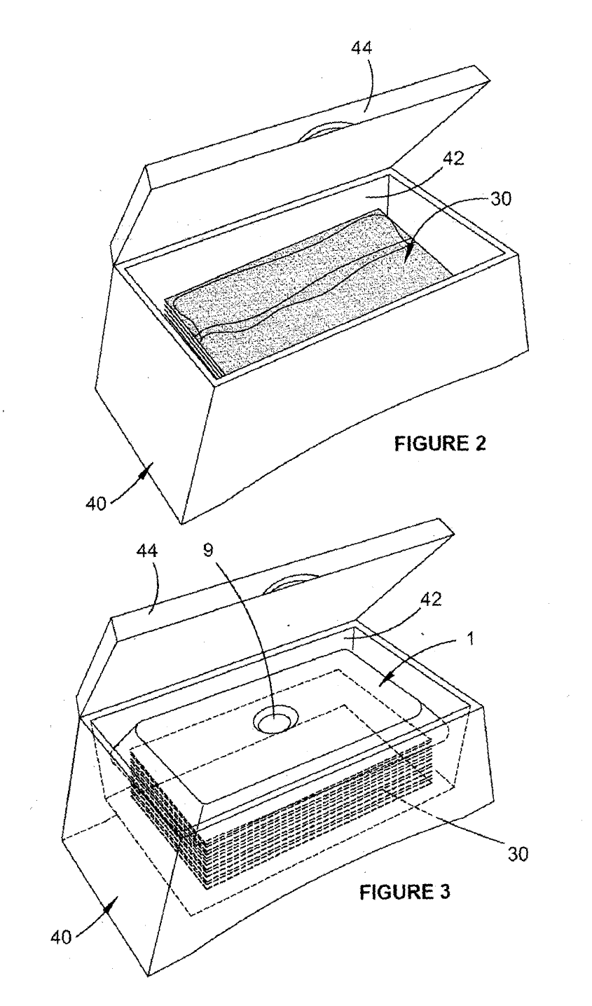

With reference to FIGS. 2 and 3, the heating device 1 is shaped for receipt against an uppermost wipe in a pile 30 of interleaved wipes in a container 40. The block 2, and more particularly the base 7, has a length and width which are such that the block 2 overlies substantially the entirety of the exposed area of the uppermost wipe. The length and width may, for example, be about 17 cm and about 10 cm respectively, consistent with the length and width of typical interleaved wipes, with the skirt 10 having a thickness of about half a centimetre, and the block 2 may have a thickness/height of about 2 cm (though it will, of course, be appreciated that various alternative dimensions are possible). The container 40 may be that in which the wipes are sold or instead one into which the wipes are transferred after being purchased, and preferably its interior is dimensioned such that there is minimal clearance between the perimeter of the device 1 and the container walls (whereby heat escape, between the device 1 and those walls, from the wipes is restricted). The container 40 includes a compartment 42, which receives the pile 30 and device 1, and a lid 44 which can be opened to provide access to the compartment 42, and closed to cover the compartment 42. The container 40 is preferably insulated such that heat escape therefrom, particularly when the lid 44 is closed so as to cover the device 1 and wipes 30 (see FIG. 5), is minimal. For example, the walls of the container may incorporate or be formed of expanded plastics foam, such as expanded polystyrene or expanded polypropylene, and/or may be configured with an evacuated void arrangement so as to be vacuum-insulated.

The device 1 is rendered ready for use by heating of the block 2, preferably (but not necessarily) with skirt 10 removed therefrom, in a microwave oven (generally for a few minutes), such that the PCM melts/softens. Following heating, the assembled device 1 (comprising the block 2 and skirt 10 fitted thereto) is placed inside the container 40, as shown in FIG. 3, atop the pile 30 therein, so as to transfer heat into the pile 30. A person handling the device 1 may grasp it via fitted skirt 10, such that his or her hand is protected from being burnt by block 2, which may be hot if only recently heated in the microwave oven.

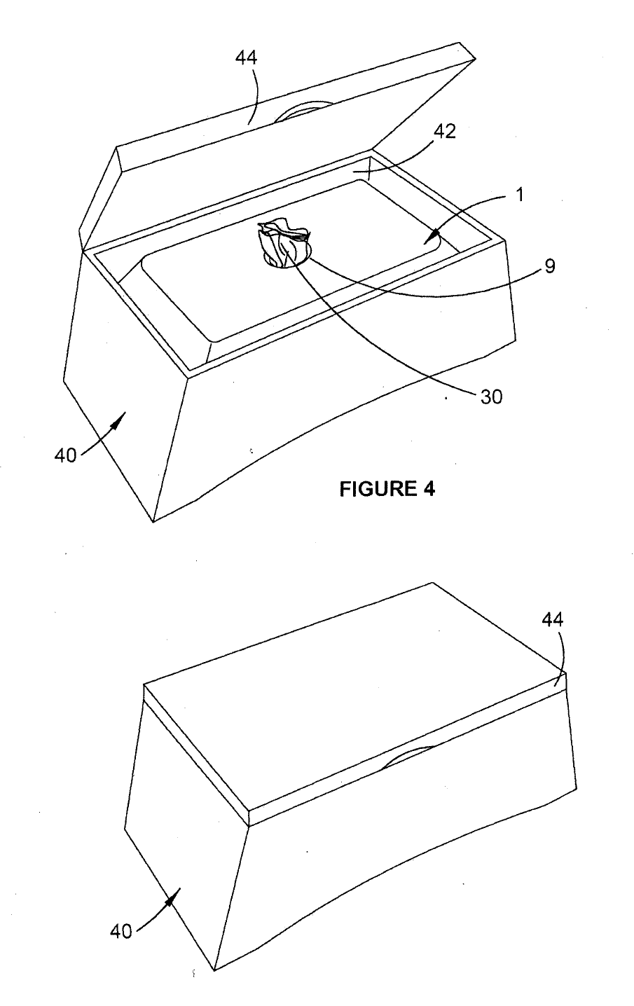

A person can insert a finger and thumb through the aperture 9 to pinch the uppermost wipe in the pile and draw it through the aperture 9, whereupon the device 1 incrementally sinks, under gravity, within the container 40 to rest against the next uppermost wipe. Owing to the interleaved relationship between the wipes in the pile 30, removal of the first uppermost wipe draws part of the subsequent uppermost wipe through the aperture 9, as shown in FIG. 4, whereby it is readily graspable for removal when required. The device 1 can then be maintained atop the pile 30 within the container 40, preferably with the lid 44 closed so as to output a continuous heat supply to the pile. The skirt 10 serves to restrict heat loss from the block 2, and its being configured so as not to cover the block base 7, whereby the base 7 directly contacts the pile 30, promotes efficient heat transfer to the pile 30.

The skirt 10, though preferable, is not essential, and may be omitted, without departure from the invention, so that the device is then constituted solely by the block 2.

In the following discussion of further embodiments of the present invention, the same reference numerals will be used to denote the features corresponding to those of the first embodiment.

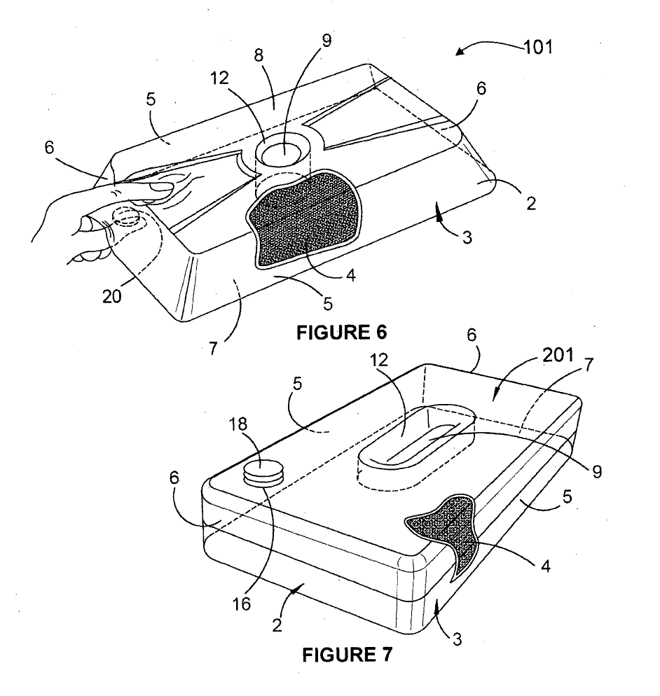

Referring to FIG. 6, a heating device 101 according to a second preferred embodiment of the invention comprises a pad or block 2 comprising a casing 3 which, consistent with the device 1, has side walls 5, end walls 6, bottom wall 7, top wall 8 and an aperture 9, and a

PCM 4 contained in the casing 3. The skirt 10 (not shown) may form part of the device 101 (in which case it fits to the block 2 in the same manner as in the first embodiment) though, again, need not. The PCM 4 in this embodiment comprises a solution containing a dissolved compound which can precipitate out of solution and in so doing liberate heat. More particularly, the solution is aqueous sodium acetate solution, which is non-toxic, having a concentration which such that the sodium acetate is fully dissolved when the block is sufficiently heated and can remain stably dissolved when the block cools to room temperature whereby the solution is supersaturated with sodium acetate when it has so cooled.

The heating device 101 further comprises an element, in the form of a non-ferrous metal disc 20, which can be flexed or otherwise deformed to effect friction between the PCM and the disc surface, thereby triggering nucleation, and thus precipitation, of the compound dissolved in the supersaturated solution, causing latent heat to be released over a sustained period As can be seen in FIG. 6, the device 101 comprises a casing, shell or skin 3 which is sufficiently flexible or pliable to allow a person to pinch walls 7 and 8 thereof against the disc 20 to effect the deformation of the latter and/or friction between it and the disc 20, and thereby trigger crystallisation of the sodium acetate and thus heating of the block/pad 2.

The device 101 is rendered ready for use by being heated, such that the compound dissolves, then allowed to cool, such that the solution is supersaturated. The device 101 may then be stored inside the container 40, consistent with FIG. 3, or kept separate from the container 40. When it becomes necessary to heat the wipes, a person manipulates the casing 3 to effect nucleation at the disc 20, triggering heat generation. The device 101 is then maintained atop the pile 30 within the container 40, so as to transfer heat into the pile 30, through the top of the pile 30. Removal of duly heated wipes 30 may then be carried out in the same manner as described for the device 101.

Because the casing 3 and disc 20 are made of microwave-safe materials, the device 101 can also be heated in a microwave oven to dissolve the sodium acetate, such that the PCM, after subsequently cooling, becomes a supersaturated sodium acetate solution once again.

Referring to FIG. 7, a heating device 201 according to a third embodiment, consistent with the device 1, comprises a block 2 comprising a casing 3 having side walls 5, end walls 6, bottom wall 7, top wall 8 and an aperture 9 (which is shown as a slot but could equally be circular, consistent with that in the first embodiment, or have any of a number of other configurations, including those referred to above), and a PCM 4 contained in the casing 3. The device 201 further comprises an inlet 16 and a plug 18 which closes the inlet 16 and is removable to provide access to the interior of the casing 3 to permit the PCM 4 to be supplied to or removed from the casing 3. The PCM 4, again, can comprise any of multiple substances without departure from the invention. However, this embodiment may be particularly useful where the PCM is one which degrades with use and must thus be replaced or replenished over time. The device 201, depending on the choice of PCM, may be one which includes a nucleation element 20 within the casing 3, in which case the casing 3 will be suitably configured to enable manipulation of the element 20, or might instead not include such an element, in which case the casing 3 could be rigid.

Again, skirt 10 (not shown) may form part of the device 201 (in which case it fits to the block 2 in the same manner as in the first embodiment) though need not.

Referring now to FIGS. 8A to 8D, a heating device 301 according to a further preferred embodiment of the invention, consistent with the previously described devices, comprises a pad or block 2 comprising a polypropylene casing 3 having side walls 5, end wall 6, bottom wall 7, top wall 8 and aperture 9 and a PCM 4 contained in the casing 3, the PCM 4 in this embodiment comprising PEG-4000. The device 301 is microwave-safe, so as to be able to be heated in a microwave oven to soften/melt the PCM 4. It has been found that a PEG-4000 content of 300 grams affords the device 301 particularly favourable characteristics. A lower part of the aperture 9 in this embodiment is downwardly divergent so as to define a cavity 11 which is bounded at a lower end thereof by the bottom wall 7, which wall is generally flat such that substantially the entirety thereof will contact the pile of wipes when the device 301 is received atop that pile. The cavity 11 will house part of the uppermost wipe in the pile which is partially drawn through the aperture 9. The upper end of the aperture 9 is configured in the form of a slot or elongated hole. The wall of the cavity 11 may provide heat to part of the uppermost wipe which is partially drawn through the aperture 9. Although the size and relative dimensions of the device can vary considerably without departure from the invention, it has been found that typical wipes sizes are such that, referring to FIG. 8D, the following dimensions afford the device favourable characteristics:
block length L: 16-18 cm;
block width W: 9-10 cm;
aperture length x: 6-7 cm;
aperture width y: 2-3 cm.

Also, the cross sectional configurations shown in FIG. 8D have been found to afford the block favourable characteristics.

Referring to FIGS. 9A and 9B, a heating device 401 according to a further preferred embodiment of the invention comprises the casing 3 and PCM 4 of the device 301 (and thus essentially the device 301 in its entirety) and insulating jacket 10, which is received over the casing 3, covering the top wall 8, side walls 5, end walls 6 and base 7, and is formed with top and bottom openings 15A, 15B which align with the aperture 9. Alternatively, the jacket may be formed from any of a range of suitable insulating materials, and may, for example, be rigid, semi-rigid or pliable. The jacket 10 may be removable from the block or permanently attached to it, e.g. via adhesive, and may be themed—e.g. it may carry an image which is pleasing to a baby or small child—and may be provided as part of a set of interchangeable jackets 10 which are themed differently/carry different images. In this embodiment, a lower wall 18 of the jacket 10, as shown in FIG. 9B, rather than the lower wall 7 of the block 2, lies in contact with the wipes when the device 401 is in use, heat being transferred from the block 2, through the lower wall 18, to the wipes.

The device 401, like the device 301, is microwave-safe, so as to be able to be heated in a microwave oven to soften/melt the PCM 4, the jacket 10, protecting the hand(s) of a person handling the device from heat output from the PCT 4.

It has been found that a Neoprene/polychloroprene cover thickness of 3 mm affords the device 401 favourable characteristics.

Referring to FIG. 10, a heating device 501 according to a sixth preferred embodiment of the invention, consistent with the previously described devices, comprises a pad or block 2 comprising a polypropylene casing 3 having side walls 5, end walls 6, bottom walls 7 and top wall 8. Again, the aperture 9 in this embodiment is downwardly divergent so as to define a cavity 11 which is bounded at a lower end thereof by the bottom wall 7, which wall is generally flat such that substantially the entirety thereof will contact the pile of wipes when the device 501 is received atop that pile, the cavity 11 being arranged to house part of the uppermost wipe in the pile which is partially drawn through the aperture 9. The upper end of the aperture 9 in this embodiment is configured in the form of a slot. The wall of the cavity 11 may, again, provide heat to part of the uppermost wipe which is partially drawn through the aperture 9. The insulating skirt, cover or jacket 10 in this embodiment is moulded, preferably in a manner such that it is fused or otherwise adhered to the casing 3, and is made of silicone. The PCM 4, again, can comprise any of multiple substances without departure from the invention, though preferably comprises PEG-4000. The device 501 is, like the previously described devices, microwave-safe, so as to be able to be heated in a microwave oven to soften/melt the PCM 4, the insulation 10, as it does in the previously described embodiments, protecting the hand(s) of a person handling the device from heat output from the PCM 4.

Referring to FIG. 11, a heating device 601 according to a seventh embodiment of the invention is identical to the device 501 except with respect to the insulating skirt/cover/jacket 10, which in this embodiment is formed of Neoprene and is received, and preferably stretched, over the casing 3 to overlie substantially the same portions thereof as in the previous embodiment, and with respect to the cavity 11, which, although being upwardly convergent, consistent with that in the previous embodiment, has a generally “dog bone-shaped” cross-sectional configuration, corresponding to the configuration assumed by a wipe as it is drawn from pile through the cavity 11/aperture 9, rather than a cross-sectional configuration shaped as a slot or as a rectangle with rounded corners.

In each of the described embodiments, the casing is, as and where appropriate, able to expand and contract sufficiently to accommodate changes in volume of the PCM during heating/cooling thereof.

In each of the embodiments described thus far, the insulation 10 may be “themed”, as mentioned above, and or carrying branding indicia.

Another example of an alternative PCM, which could be instead used in any of the previously described embodiments (with element 20 omitted), is paraffin wax, which, advantageously, is chemically inert and non-toxic, and is readily available at a low cost, and undergoes only a slight change in volume in changing phase (of approximately 10%). Such was may have a melting point which is generally between 48° C. and 62° C.

Other PCMs suitable as substitutes in either embodiment may comprise, for example, an inorganic PCM, such as a salt hydrate (N_{N}H₂O), an organic PCM, such as a fatty acid PCM, or a eutectic, comprising an organic-organic, organic-inorganic or inorganic-inorganic compound. Particular ones of these alternatives, including in particular organic PCMs (including paraffin wax) are “self-nucleating” so that they do not require a nucleation site defined by a disc or other suitable element (whereby, in such embodiments, disc 20 can be omitted).

Heating devices somewhat analogous to those described above may hold a heating material which is not a PCM, without departure from the invention. One such heating device 701 according to a further embodiment, which is illustrated in FIG. 12, comprises a bag 50 and, like the devices previously described, is configured to rest against the pile of wipes 30 in the container 40, and formed with an aperture 9 therethrough, exending between lower and upper surfaces thereof, through which the wipes can be drawn one after another. The bag 50 may hold heating material comprising particulate or granular matter, such as seeds, wheat grains, uncooked rice, feed corn, buckwheat hulls and/or barley beans, all of which are microwave-safe and would allow for the device as incorporating them to be heated (with the disc 20 then, of course, being omitted). The bag may, but need not necessarily, be air/moisture permeable. In such an embodiment, the device would need to be heated and, while still hot, placed and maintained atop the wipes. A casing, which may be the same as casing 3 in any of the previous embodiments, may be employed in place of the bag 50. The device 100 may, but for its being configured with aperture 9, be scented—e.g. it may contain lavender or another substance having an odour, in addition to or constituting the heating material—so as to impart a scent or fragrance to the wipes.

Advantageously, the devices according to the preferred embodiments of the invention, in being microwave-safe, can be simply and quickly heated wherever a microwave oven is on-hand.

Advantageously, the devices according to the preferred embodiments of the invention are received directly against the towelette which is to be the first in the layered arrangement of towelettes to be used, whereby heating is reliable and efficient.

In each of the embodiments, the device is of sufficient weight to prevent it being lifted appreciably from the pile of wipes by the uppermost wipe as it is drawn therethrough.

While various embodiments of the present invention have been described above, it should be understood that they have been presented by way of example only, and not by way of limitation. It will be apparent to a person skilled in the relevant art that various changes in form and detail can be made therein without departing from the spirit and scope of the invention. Thus, the present invention should not be limited by any of the above described exemplary embodiments. For example, features from different ones of the embodiments described and illustrated may be combined without departure from the invention.

Throughout this specification and the claims which follow, unless the context requires otherwise, the word “comprise”, and variations such as “comprises” and “comprising”, will be understood to imply the inclusion of a stated integer or step or group of integers or steps but not the exclusion of any other integer or step or group of integers or steps.

The reference in this specification to any prior publication (or information derived from it), or to any matter which is known, is not, and should not be taken as an acknowledgment or admission or any form of suggestion that that prior publication (or information derived from it) or known matter forms part of the common general knowledge in the field of endeavour to which this specification relates.

1. A device for warming wipes, comprising a body configured to rest atop a pile of wipes so as to impart heat thereto, and being configured with an opening therethrough, through which successive uppermost wipes in the pile can be drawn.

2. A device according to claim 1, wherein the opening comprises an aperture.

3. A device according to claim 1, wherein the body is configured in the form of a block.

4. (canceled)

5. A device according to claim 1, wherein the body comprises a covering and a substance, contained in the covering, which can be heated so as to be able to output heat for warming the wipes.

6. (canceled)

7. A device according to claim 5, wherein the covering comprises a casing.

8. (canceled)

9. A device according to claim 5, wherein the substance comprises a phase change material.

10. (canceled)

11. A device according to claim 9, wherein the phase change material comprises a solution containing a dissolved compound which can precipitate out of the solution and in so doing liberate heat, having a concentration which such that compound is fully dissolved when the body is sufficiently heated and can remain stably dissolved when the body cools to room temperature whereby the solution is supersaturated with the compound, and wherein the body includes means to effect nucleation of the compound.

12. (canceled)

13. A device according to claim 11, wherein the phase change material comprises an aqueous sodium acetate solution.

14.-15. (canceled)

16. A device according to claim 1, further comprising an insulating jacket or skirt received over the body to restrict heat loss therefrom.

17. A device according to claim 16, wherein the insulating jacket or skirt is configured such that a lower face of the body is exposed to contact directly the uppermost wipe in the pile.

18. A device according to any claim 1, wherein the body is heatable in a microwave oven.

19. A device according to claim 1, being heatable in a microwave oven.

20. (canceled)

21. An apparatus for storing and warming wipes, comprising:
a receptacle for holding a pile of wipes; and
a device according to claim 1 receivable atop the pile in the receptacle,
wherein the receptacle is configured to permit the successive uppermost wipes to be drawn through the opening and removed from the receptacle when the device is received atop the pile in the receptacle.

22. An apparatus according to claim 21, further comprising said pile of wipes held in the receptacle, wherein said device is arranged atop the pile.

23. An apparatus according to claim 22, wherein the wipes are interleaved whereby withdrawal of an uppermost wipe in the pile through the opening effects drawing of part of the subsequent uppermost wipe through the opening.

24.-25. (canceled)
